(19) United States
(12) Patent Application Publication
(10) Pub. No.: US 2003/0203006 A1
(43) Pub. Date: Oct. 30, 2003

(54) Immunomodulator

(75) Inventors: Junji Hamuro, Kawasaki-shi (JP); Yukie Murata, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: AJINOMOTO CO. INC., Tokyo (JP)

(21) Appl. No.: 10/405,506

(22) Filed: Apr. 3, 2003

Related U.S. Application Data
(62) Division of application No. 09/181,881, filed on Oct. 29, 1998, now abandoned.

(30) Foreign Application Priority Data
Oct. 29, 1997 (JP) 312727/1997

(51) Int. Cl. A61K 38/05; A61K 31/7048; A61K 31/385; A61K 31/353

(52) U.S. Cl. 424/439; 514/18; 514/440; 514/27; 514/456

(57) ABSTRACT

An immunomodulator is provided which is capable of oral intake for improvement, treatment and prevention of human immunological diseases and which is used to treat, improve and prevent human immunological diseases, especially, autoimmune diseases and allergic diseases such as hepatic cirrhosis, hepatitis, diabetes, inflammatory bowel diseases, chronic rheumatoid arthritis, asthma and cutaneous atopy, allergic diseases and cancers by a new method that can control the redox state of macrophages or monocytes, and can be incorporated into a drug, a food, a nutrient, and an infusion. The contents of oxidative glutathione and reductive glutathione in macrophages are monitored, and the ratio of oxidative glutathione and reductive glutathione is examined, whereby macrophages are classified into oxidative macrophages and reductive macrophages having different functions. The degree of progression of various autoimmune diseases is analyzed from this standpoint. On the basis of the results, an immunomodulator capable of oral intake which contains a substance having an activity of changing a content of reductive glutathione in macrophages to solve the above-mentioned problem and which is intended to treat, improve and prevent human immune diseases is provided.

### BACKGROUND OF THE INVENTION

1. Field of the Invention

The present invention relates to a novel immunomodulator. More specifically, the present invention relates to an immunomodulator capable of oral intake which has a novel suppressive function of macrophages (hereinafter sometimes abbreviated as “MΦ”) or monocytes. The immunomodulator may be used for treatment, improvement and prevention of human autoimmune diseases such as hepatic cirrhosis, hepatitis, diabetes, inflammatory bowel diseases, chronic rheumatoid arthritis, asthma and cutaneous atopy, allergic diseases and cancers. The present invention also relates to a drug, a food, a nutrient and an infusion containing the immunomodulator.

2. Description of Related Art

As used herein, the term “immune system” refers to one's bodily system for defending itself from exogenous infection by virus, bacteria or the like, or from invasion the body with transformed cells (tumor cells and the like) formed by transformation of autologous cells. However, the immune system occasionally behaves abnormally, i.e., it functions excessively and acts to reject autologous components. On the other hand, the immune system sometimes functions deficiently, resulting in an immunocompromised state. Diseases revealing these abnormal responses are generally called immunological diseases. Examples thereof include diverse diseases, such as, for example, acute or chronic inflammatory diseases such as atopic cutaneous inflammatory diseases, pollinosis, asthma and sarcoidosis; autoimmune diseases such as allergic diseases, chronic rheumatoid arthritis, diabetes, SLE and chronic fatigue syndrome; hepatitis, hepatic cirrhosis, inflammatory bowel diseases (IBD) such as ulcerative colitis and Crohn's disease; and cancer cachexia . These immunological diseases originate from complex pathological causes. Systemic immunodeficiency and functional deficiency originate from pathological inflammation accompanied by cell proliferation, differentiation or cell necrosis through local production of cytokines or inflammatory mediators.

As cells that participate in immunity, T lymphocytes and B lymphocytes are well known, exhibiting a wide variety of functions as cells playing roles in cellular immunity and humoral immunity respectively. Meanwhile, macrophages and monocytes are cells that intimately participate in both cellular immunity and humoral immunity, and they intimately participate in rejection of non-self foreign bodies, for example, immunological diseases such as allergy and rheumatism, cancers and bacterial infection.

The functions of macrophages and monocytes are classified into the following four types: a secretary function, an immunomodulatory function (mainly antigen presentation), treatment of foreign bodies and waste matters, a phagocytic function and a cytotoxic/cytostatic activity against target cells. It is widely accepted that these cells produce diverse inflammatory mediators. For example cytokines such as TNF, IL-12, IL-1, IL-6, TGFβ and IL-8 and so on; hormonal molecules such as neopterine (NPT) and dihydroxyepiandrosterone (DHEA); arachidonic acid metabolites such as PGE2 and LTB4; complement related molecules such as C5a and C3; such as reactive oxygen and reactive nitrogen intermediates. It has not been clarified whether these diverse functions are exhibited by one kind of macrophage or monocyte or by distinctive groups of macrophages or monocytes having different functions. While lymphocytes are classified into distinctive subsets according to their cell surface markers and the distinctive function markers uniquely correspond to each subset of lymphocyte, the correspondence between the wide variety of functions of macrophages is less clear. Monocytes have not been classified into cellular subsets. For this reason, although macrophages and monocytes play quite important roles in the triggering and the pathological progression of the above mentioned inflammatory, allergic and immunological diseases, the functional classification of macrophages and monocytes subsets has not yet been applied at all to therapeutic, prophylactic and preventive treatment of human diseases, with the assumption of the presence of macrophage and monocyte subsets, and even the hypothesis thereof has not yet been given.

In recent years, in the patients suffering from allergic diseases, autoimmune diseases such as chronic rheumatoid arthritis and cancer, the inclination of helper T cell subsets in the peripheral blood has been pointed out and has been linked to the pathology of these diseases. Helper T lymphocytes which are a subset of T lymphocytes have been further classified into two subsets, namely Th1 and Th2, and it now appears that the ratio of these two types is an relevant index of immunological functions of patients. Attempts are being made to establish a more appropriate therapeutic treatment by diagnosis of the ratio or by improvement of the ratio based on this index. That is, it is known that when the amount of Th2 inducing IgE production from B cells is higher than that of Th1 (Th1<Th2), allergic diseases are worsened. Attempts are being made to suppress allergy upon measuring a Th1/Th2 ratio to examine an immunological responses of patients or to provide Th1 response superior to Th2 responses. On the contrary, the presence of diseases caused by a predominance of Th1 has been successively indicated also in chronic rheumatoid arthritis or an asthmatic inflammatory disease at the chronic stage.

Even when the Th1/Th2 balance is measured using biological materials and the functions of the two subsets are modulated , this modulation has not successfully been utilized currently in the examination or the diagnosis of local chronic inflammatory diseases or allergic diseases. The terms such as Th1 diseases and Th2 diseases have been lately used. However, these terms cannot necessarily be distinguished clearly.

The Th1/Th2 presence ratio is a mere index of lymphocyte subsets. Since the in vivo dynamism of the lymphocyte subsets is actually regulated by the cell group called accessory cells including macrophages in the present invention, it is difficult to appropriately diagnose the progression of diseases with only the Th1/Th2 presence ratio and to treat the same on the basis of this index. As will be described below, the Th1/Th2 balance is controlled by the distinct macrophage/monocytes functions. Even if a skewing to Th1>Th2 is intended, this is hardly effective for therapy of immunological diseases, due to the presence of a complex cytokine network, and a new index for diagnosis and therapy has been in demand.

It has been clarified that in macrophages intimately participating in the inflammatory reactions, the functions of the cells are variable depending environmental factors such as oxidative stress, cytokine stimulation, infection with virus or bacteria, and the like. However, the correspondence between the functions and the classification of cell subsets of macrophages is unknown. New findings are required in the above-mentioned classification of functions and subsets, and these findings will lead to the development of quite useful new therapeutic methods. Under such circumstances, the development of effective agents for modulating immunity, namely, immunomodulators, has been in demand.

### SUMMARY OF THE INVENTION

The present inventors have assiduously conducted investigations to solve the above-mentioned problems, and have consequently found the following findings. That is, they have attempted to distinguish macrophages (including monocytes) which share with an immunosuppressive activity, a cachexia inducting activity, an activity of inducing malignant progression and an activity of prolonging inflammation from immunomodulatory macrophages in view of a difference in a redox state (potential) of macrophages, and have then succeeded in this attempt. The reductive glutathione (GSH) content in macrophage cells is employed as an index thereof.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a diagrammatic view showing a relationship of a difference in a function of macrophages, with respect to the Th1 and Th2 balance, immunosuppression, malignant progression, cancer cachexia, and local inflammatory responses.

FIG. 2 illustrates that the presence ratio of oxidative and reductive macrophages controls the immunological functions through the skewed generation of Th1 and Th2 cytokines. This is based on the new findings of the present inventors, showing that the redox condition of macrophages plays an important role in amplifying the inclination of the in vivo responses between humoral and cellular immunity.

FIG. 3 is a view showing the results of the examination of functions of both macrophages, namely the functional differences between oxidative macrophages and reductive macrophages.

FIG. 4 is a view showing the results of examining whether there is a difference in the IL-12 production between Lentinan (LNT) induced MΦ and Lipopolysaccharide (LPS) induced MΦ. It indicates that there is a great difference in the amount of IL-12 (Th1 cytokine) produced between oxidative and reductive macrophages and IL-12 is produced only from reductive macrophages with the high cellular reductive glutathione content.

### DETAILED DESCRIPTION OF THE INVENTION

Glutathione is present in all mammalian cells, and well known as an intrinsic antioxidant. It is a tripeptide having a wide variety of functions in cells, such as removal of radicals or peroxides, metabolism of eicosanoids such as prostaglandin, detoxication of biologically foreign materials, amino acid transport and the like. Glutathione includes reductive glutathione (GSH) and oxidative glutathione (GSSG), and these form a conjugate cycle. In normal cells, the content of reductive glutathione (GSH) is higher, and it acts defensively on oxidative stress, especially on H₂O₂.

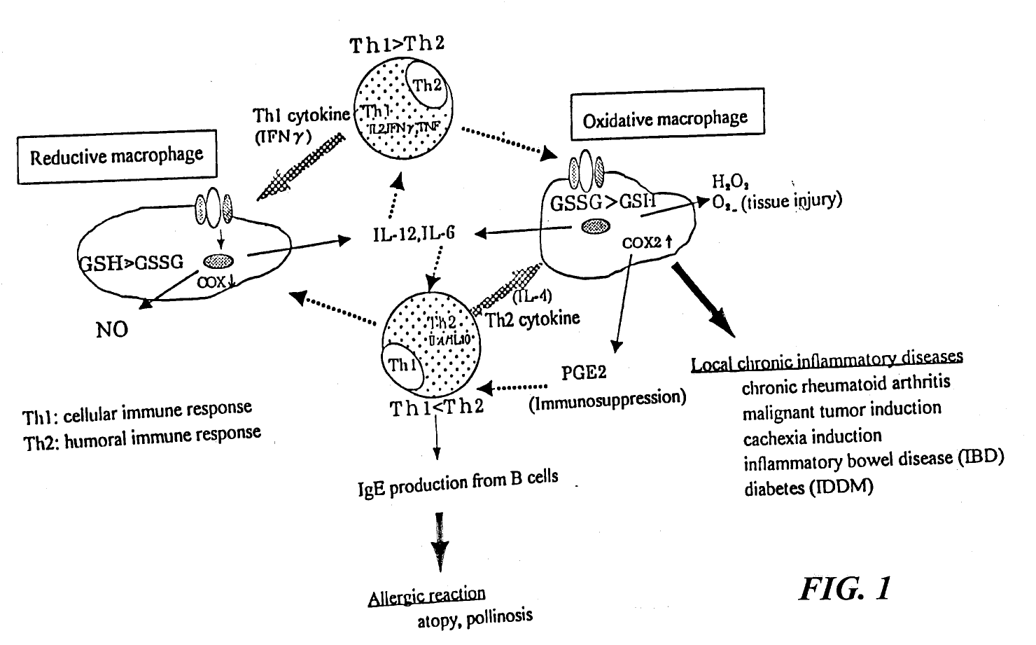

Ruede et al. have already reported that with respect to macrophages differentiated in the presence of GM-CSF and macrophages differentiated in the presence of M-CSF from monocytes, the cellular GSH content of the former is higher than that of the latter, so that the difference in the GSH content in cells seems likely to participate in the function of macrophages (Germann, T., Mattner, F., Partenheimer, A. et al.: Different accessory function for Th1 cells of bone marrow-derived macrophages cultured in granulocyte-macrophage colony stimulating factor or macrophage colony-stimulating factor, Int. Immunol., 4:755, 1992; Frosch, S., Bonifas, U., Eck, H.-P. et al. : The efficient bovine insulin presentation capacity of bone marrow-derived macrophages activated by granulocyte macrophage colony-stimulating factor correlates with a high level of intracellular reducing thiols, see Eur. J. Immunol., 23; 430, 1993). The present inventors have measured the reductive GSH content in macrophages, and have found that there is a great difference in an immunological function between macrophages having different GSH contents (refer to FIG. 1); they have tested the immune responses with regards to the cellular GSH content, and have found that the redox states can artificially be modulated with an orally administerable low-molecular substance, and that these substances capable of modulate intracellular GSH content can widely be applied to treatment of wide variety of diseases, and the substance can be also used as a food (refer to FIG. 1). These findings have led to the completion of the present invention.

FIG. 1 is a diagrammatic view showing a linkage between a difference in a function of macrophages or monocytes (both are referred to as “macrophages” in the present invention), and an effect on a Th1/Th2 balance, a mechanism of immunosuppression, cachexia induction and induction of malignant tumor progression caused by a functional difference of macrophages and local inflammatory diseases. For example, according to the tumor bearing progression, the local-Th1/Th2 balance is skewed, an inclination to humoral immunity appears, the structure and the function of the cytokine receptor complex are changed, oxidative macrophages with a low intracellular GSH content are increased, the production of active oxygen or inflammatory mediators such as PGE2 and IL-6 are increased to cause systemic immunosuppression or induction of cachexia and to prolong chronic inflammation accompanied by allergic reactions or tissue injury.

The present inventors have conducted further investigations on the basis of the above-mentioned findings, and have consequently found that functionally heterogeneous macrophages, which play important roles in the inflammation reactions can be classified into two groups, namely, oxidative macrophages and reductive macrophages by determining the cellular content of oxidative glutathione and the cellular content of reductive glutathione in macrophage cells. The oxidative macrophages induce local chronic inflammatory diseases or an allergic reaction in immune diseases and the Th1/Th2 balance controlling the balance of humoral and cellular immunity is regulated with the redox state of macrophages, that the redox state of the macrophages plays an important role in immunological diseases, and this redox state is monitored and artificially controlled or modified which is useful in the diagnosis or the therapy of these immunological diseases, and that this control can easily be conducted using low-molecular weight substances capable of oral intake.

With respect to the definition of the oxidative macrophage and the reductive macrophage in the present invention, macrophages are reacted with monochlorobimane which is a chemical reagent specific to reductive glutathione (GSH) to determine the GSH content in cells. The macrophage of which the GSH content is increased in comparison with the resident macrophage is defined as a reductive macrophage, and the macrophage of which the GSH content is decreased is defined as an oxidative macrophage. Further, the macrophage of which the GSH content becomes more than 2 nmoles/5×10⁵ macrophage cells by bringing a low-molecular weight substance capable of oral intake into contact with the macrophage for from 2 to 24 hours is defined as the reductive macrophage (or monocyte), and the macrophage of which the GSH content becomes less than 0.1 nmoles/5×10⁵ macrophage cells is defined as the oxidative macrophage. Alternatively, the macrophage of which the GSH content is two or more times than that of the resident macrophage is defined as the reductive macrophage, and the macrophage of which the GSH content is ⅕ or less that of the resident macrophage is defined as the oxidative macrophage.

At present, it is considered that the Th1/Th2 balance is regulated by the ratio of IL-6 or IL-4 and IL-12 produced in vivo. It has been already known that Th2 participating in humoral immunity is induced by the former two and Th1 by IL-12 respectively. It is clarified that IL-6 and IL-12 are produced from macrophages. However, assuming that the same macrophage cells produce both IL-6 and IL-12, one type of a macrophage participating in both the Th1 induction and the Th2 induction comes to be present. Thus, there is a great contradiction in considering the host immune responses.

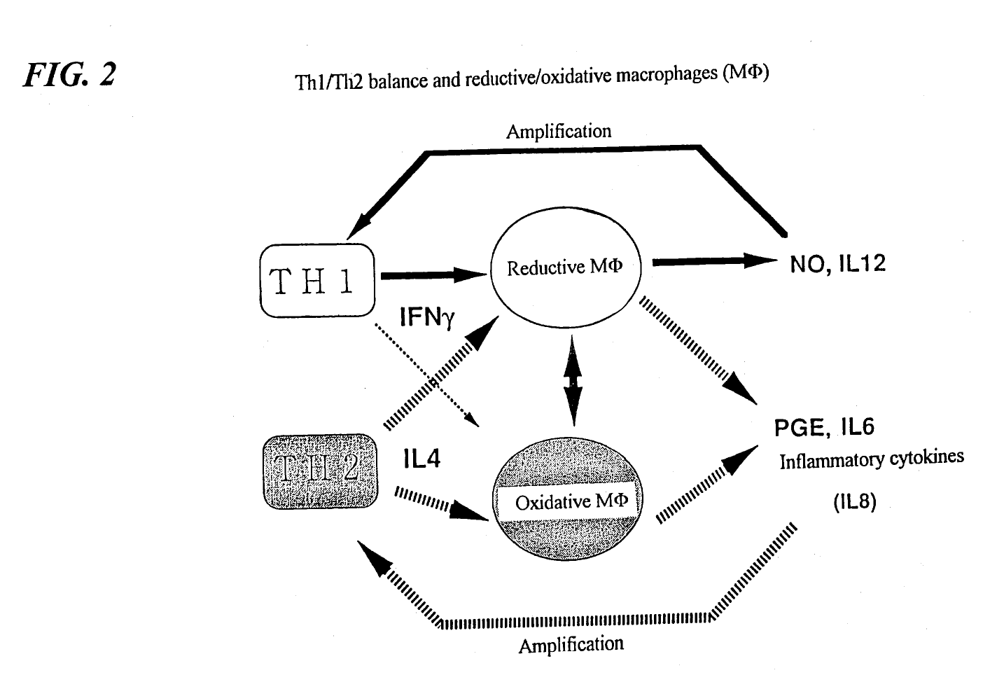

The present inventors have found that IL-12 is produced from only the reductive macrophage having the high intracellular GSH content to act on the Th1 induction and that the IL-6 production is increased in the oxidative macrophage to induce Th2. They have further found that when the macrophage is inclined to the oxidative type in spite of the production of IFNγ, a typical Th1 cytokine, IL-6 skewing the balance to Th2 is produced in a large amount by IFNγ stimulation. On the contrary, it has also been found that IFNγ, a typical Th1 cytokine, increases the phenotype of the reductive macrophage further by acting on the reductive macrophage. When IL-4, the typical Th2 cytokine, acts on the oxidative macrophage, the oxidative macrophage phenotypes are further increased. This knowledge indicates that the balance between humoral immunity and cellular immunity is unequivocally defined by the redox state of macrophages, and they are relevant new findings innovating the basic concept of immunology (refer to FIG. 2). On the basis of these findings, the quite useful, original invention overcoming the conventional confused immunological disease therapy was already completed with respect to the diagnosis and the therapy of the immunological diseases. Investigations have been assiduously conducted on the basis of the above-mentioned findings. Consequently, the present invention has been completed.

That is, the present invention is an immunomodulator containing substances having an activity of changing the content of glutathione in macrophages. The macrophage also includes monocytes. This substance is preferably one which provide MΦ with productivity of interleukin 12 by increasing the content of reductive glutathione in macrophages. More preferable examples thereof include low-molecular weight substances, for example, a GSH precursor metabolized in GSH within cells, such as N-acetylcysteine (NAC); glutathione derivatives such as glutathione monoester and glutathione diester; lipoic acid and derivatives thereof, and ortene. These can be administered orally or percutaneously. It is also possible to use antioxidants such as flavonoid and derivatives thereof which raise the GSH content, increase the production of IL-12 and decrease the production of IL-6 by contact with macrophages. Further, high-molecular weight substances which are used in combination therewith, such as β (1-3) glucan and cytokine, are preferably used in the intravenous administration and the administration using DDS (drug delivery system). Preferable examples of the cytokine include IL-4, IL-2, IL-12, TGFβ and IFNγ. When it is required to increase cellular immunity, IL-2 and/or IFNγ is especially preferable. When it is required to decrease cellular immunity, IL-4 and/or TGFβ is especially preferable. These substances can be contained either solely or in combination, and a higher effect is expected by a combination of a low-molecular orally administerable immunomodulator and a high-molecular immunomodulator suited for intravenous administration.

Further, the present invention also includes an immunomodulator containing a substance which can selectively remove either of two types of macrophages, reductive macrophages or oxidative macrophages which are different in the intracellular content of reductive glutathione. Examples of the substance include a substance in which a cytotoxic DNA alkylating agent is conjugated with glutathione, and a substance in which an oxidative or reductive macrophage-specific antibody is conjugated directly or through a linker with a low-molecular weight compound having a cytotoxicity to macrophages with a material showing a cytotoxicity after being incorporated into a macrophage. Examples of the alkylating agent include cyclophosphamide, nimustine (ACNU), mitomycin C and melphalan. In an oxidative macrophage in which glutathione S-transferase is activated, an DNA alkylating agent, bound to glutathione directly or through a linker, is deconjugated by the action of this enzyme and can remove the reductive macrophage by specifically killing the same. Further, a substance which has no cell-killing property in vitro but comes to show the cell-killing property with the action of an enzyme increased in oxidative or reductive macrophages can also be used as a prodrug.

Further, the present invention includes a food, a nutrient or an infusion containing the above-mentioned immunomodulator. The food includes ordinary foods and those which are put into the mouth, such as a toothpaste, a chewing gum and the like. It is especially preferable to incorporate the immunomodulator in health foods. Further, it may be used as an additive which is added to a food. As the nutrient, vitamin preparations and calcium preparations are available. As the infusion, a high calorie infusion, a physiological saline solution and blood preparations are available.

In addition, the immunomodulator, the food, the nutrient and the infusion of the present invention is preferably used for improvement of the cachectic condition of patients suffering from cancers and for diabetes, inflammatory bowel diseases, chronic rheumatoid arthritis, hepatitis, hepatic cirrhosis and/or cancers chemoprevention.

Accordingly, it is an object of the present invention is to provide an immunomodulator useful for therapy of patients suffering from immunological diseases in which macrophages are classified into oxidative macrophages and reductive macrophages having different functions by determining the contents of oxidative glutathione and/or reductive glutathione in macrophage cells using a body fluid or a cell sample separated and collected from humans, and the ratio of these macrophages present is artificially controlled with a substance capable of oral intake or either oxidative macrophages or reductive macrophages are artificially removed, as well as to a food, a nutrient and an infusion which are useful for improvement of diseases.

Glutathione in the present invention is also called S-L-glutamyl-L-cysteinylglycine. It is an SH-compound which is mostly present in vivo, and generally referred to as “GSH”, Glutathione is classified into reductive glutathione and oxidative glutathione. Reductive glutathione refers to the above-mentioned glutathione (GSH). Oxidative glutathione is also called glutathione disulfide, and is referred to as “GSSG”.

The macrophage in the present invention also includes the above-mentioned monocyte. The macrophage is known to secrete or release various mediators such as cytokines and inflammatory mediators from cells thereof. Whether they are secreted or not is determined depending on its activated or differentiated condition, and the amount released varies depending thereon. In the present invention, attention is directed to the contents of oxidative glutathione and reductive glutathione in macrophage cells. Macrophages are monitored by the ratio of oxidative macrophages and reductive macrophages, and the immunological state is identified. The balance of these macrophages is modulated with the immunomodulator and the like of the present invention to improve the in vivo immunological state and to treat or prevent various diseases usefully.

In the reductive macrophage, the content of reductive glutathione is relatively higher than that in oxidative macrophage. In the oxidative macrophage, the content of reductive glutathione is relatively lower than that in reductive macrophage. Further, the reductive macrophage and the oxidative macrophage are different in activation of a transcription factors due to the difference in the reductive GSH content. Consequently, there occurs a difference in the gene expression of cytokines or inflammatory mediators, so that the type and the amount of the resulting inflammatory cytokines or inflammatory mediators are changed and the quality of inflammation is changed.

With the oxidative macrophage, inflammatory cytokines and mediators such as IL-6, IL-1, IL-8, IL-10, TNF, hydrogen peroxide, superoxide and PGE2 are produced. With the reductive macrophage, nitrogen monoxide (NO), IL-12 and LTB4 are produced. Further, the oxidative macrophage and the reductive macrophage are inter-converted through stimulation or the like. The reductive macrophage can be converted to the oxidative macrophage through artificial stimulation using LPS or PMA inducing inflammatory or ischemic shock and cytokines such as IL-4 and TGFβ. On the contrary, the oxidative macrophage can be converted to the reductive macrophage with the addition of IFNγ, IL-2, lentinan (LNT) which is an antitumor polysaccharide, or lipoic acid with an antioxidant nature. This can be applied to therapy of immunological diseases.

The amounts of the oxidative macrophage and the reductive macrophage vary depending on the pathological state of the diseases. The amount of the oxidative macrophage contained in the body fluid or the cell sample collected from patients suffering from allergic diseases or advanced cancers is relatively larger than that in the healthy person. This can be used in the examination for diagnosis of immune diseases and tumor cachexia and the subsequent therapy thereof.

When the amount of the oxidative macrophage is relatively larger than that of the oxidative macrophage in the healthy person, it is necessary to conduct the treatment for converting the oxidative macrophage to the reductive macrophage in order to improve the condition of the disease of the patient.

In accordance with the present invention, the low molecular weight compound which has an activity of changing the content of reductive glutathione in the macrophage cell after measuring the same by the above-mentioned method and which maintains the activity even through the oral intake is formulated into a drug in a usual manner, and this drug can be taken in the patient every day or at fixed intervals upon monitoring the condition of the disease. At the chronic stage, the marked effect is brought forth by the long-term administration.

With respect to the definition of the oxidative macrophage and the reductive macrophage in the present invention, the reductive glutathione (GSH) content in the cell is determined through the reaction with monochlorobimane which is a chemical reagent specific to GSH. The macrophage of which the GSH content is increased in comparison with the resident macrophage is defined as the reductive macrophage, while the macrophage of which the GSH content is decreased is defined as the oxidative macrophage. Preferably, the macrophage of which the GSH content is more than 2 nmoles/5×10⁵ macrophage cells by being brought into contact with the low-molecular weight substance capable of oral intake for from 2 to 24 hours is defined as the reductive macrophage, and the macrophage of which the GSH content is less than 0.1 nrnoles/5×10⁵ macrophage cells is defined as the oxidative macrophage. Alternatively, the macrophage of which the GSH content is at least twice that of the resident macrophage is defined as the reductive macrophage, while the macrophage of which the GSH content is at most ⅕ that of the resident macrophage is defined as the oxidative macrophage.

As the substance having the activity of changing the content of reductive glutathione in macrophage cells, any substance will do if macrophages (or monocytes) are incubated at concentrations of 5×10⁵ cells/200 μl/well using a 96-well microplate, from 0.01 μM to 5 mM of a substance to be tested are added thereto and incubated at 37° C. in a 5% CO₂ incubator and the reductive GSH content is increased or decreased relative to the control group after from 2 to 24 hours. A substance that can increase the GSH content to 2 nmoles/5×10⁵ macrophage cells or more or decrease the same to 0.1 nmoles/5×10⁵ macrophage cells or less is preferable. Examples thereof include antioxidants, for example, a precursor of GSH which is metabolized into GSH in cells, such as N-acetylcysteine (NAC), glutathione derivatives such as glutathione monoester and glutathione diester, lipoic acid and derivatives thereof, ortene, and flavonoid and derivatives thereof. They are substances having an activity of changing the content of glutathione in cells by the incubation with macrophages in vitro for a few hours. These agents can be used either singly or in combination. The effect thereof can be measured by collecting monocytes from an body fluid of local inflammatory sites or a peripheral blood after the intake or the administration and determining the change in the content of reductive glutathione in cells relative to that before the treatment by the above-mentioned method. The usefulness as the immunomodulator is clearly evaluated by this procedure, and the agents are effective for the treatment of the patients.

The agents can widely be applied to diseases associated with the abnormal Th1/Th2 balance or the functional deficiency of macrophages, for example, cachexia of patents suffering from cancers, diabetes, chronic rheumatoid arthritis, autoimmune diseases such as SLE, chronic inflammatory diseases such as hepatitis, hepatic cirrhosis, inflammatory bowel diseases, and allergic diseases such as asthma, cutaneous atopy and sarcoidosis. The agents are also effective for chemoprevention of cancers as the immunomodulator. This makes it clear that during the period in which one normal cell undergoes transformation and carcinogenesis in the human body and then reaches to 10⁹ cells where the presence of cancer tissues is clinically detected, the cancerous tissue is profitably present in the reductive condition. That is, it is scientifically verified that active oxygen or the like which is produced by inflammatory responses in vivo contributes to the malignant progression.

The immunomodulator used in the present invention can be administered singly in the actual medical care. The immunomodulators capable of oral intake which are included in the present invention can also be used in combination. Further, the immunomodulator of the present invention can be mixed with, or used in combination with, the other immunomodulator incapable of oral intake but changing the content of reductive glutathione in macrophage cells with the different function, for example, exogenous and endogenous substances such as β (1-3) glucan typified by lentinan and cytokines typified by interleukin 2 (IL-2). Especially when it is required to increase cellular immunity, IL-2 or γ-interferon (γIFN) is used in combination whereby interleukin 12 (IL-12) is produced in vivo in a large amount from the reductive macrophage to more increase the effect of the present invention. On the other hand, when the therapeutic effect is intended by decreasing cellular immunity, the production of IL-12 is decreased with the combined use of interleukin 4 (IL-4) or TGFβ to increase the effect. It has been found in the present invention that these cytokines change themselves the content of reductive glutathione in macrophage cells, increasing the usefulness and the scope of the present invention.

It is also included in the present invention that either of the macrophages which are different in the content of reductive glutathione in cells, namely, the macrophage (oxidative macrophage) having the low reductive GSH content and the macrophage (reductive macrophage) having the high reductive GSH content is selectively removed. The substance used in this case may be a low-molecular weight compound or a high-molecular weight compound. Among others, antibodies and derivatives thereof are effective.

As already stated, the correspondence of a variety of functions of macrophages/monocytes to their subsets has been to date totally unknown. Accordingly, although macrophages/monocytes play quite an important role in the triggering and the progression of inflammatory diseases, allergic diseases and immunological diseases, the functional classification on the basis of the presence of distinct macrophage/monocyte subsets has not been applied at all to the therapy, improvement and prevention of human diseases, and this application has not been even imagined. Before the completion of the present invention, the reductive GSH content of the macrophage was measured, and it was discovered for the first time that there is a great difference in an effect of macrophages having different GSH contents on the immunological functions. Further, the contents of oxidative glutathione and reductive glutathione in macrophage cells which play an important role in the inflammatory reaction were measured to classify heterogeneous macrophages into the two types, namely, oxidative macrophages and reductive macrophages. Then, it was found that the oxidative macrophages induce local chronic inflammatory diseases or allergic reaction accompanied by immunological diseases, that the Th1/Th2 balance controlling the balance of humoral immunity and cellular immunity is regulated by the redox state of macrophages, and that the redox state of the macrophages plays an important role in the progression of immunological diseases. In order to artificially control the presence ratio of these two macrophages, the above-mentioned low-molecular weight substance capable of oral intake is used as a drug, and also the selective removal of either of these macrophages is also quite useful. This is also understandable from the fact that various monoclonal antibodies to lymphocytes are on the market as an immunosuppressor. It is easily conceivable to those skilled in the art that antibodies to either of these macrophages or to markers expressed in larger amounts in either of these macrophages can be used.

Further, substances having a toxicity to cells or derivatives thereof can be used. However, since there is a great difference in intracellular enzymatic activities between reductive macrophages and oxidative macrophages, substances which can be converted to those having a selective cytotoxicity within either of reductive macrophages or oxidative macrophages are most appropriate prodrugs in the present invention. For example, the use of a pyrimidine nucleotide phosphorylase enzymatic activity or a glutathione-S-transferase enzymatic activity which is increased in the oxidative macrophages is mentioned. There is a product in which an alkylating agent having a cytotoxicity is conjugated with glutathione.

That the immunomodulator of the present invention can be applied to a wide variety of immunological diseases is clearly seen from the fact that it controls the secretion of an inflammatory mediator from macrophages at the very beginning stage of the production. For example, non-steroidal acidic anti-inflammatory drug (aspirin or the like) is said to exhibit the pharmaceutical effect by controlling production or isolation of prostaglandin. Meanwhile, an antioxidant such as vitamin E exhibits the pharmaceutical effect by controlling production of active oxygen. Thus, the function is only to control one of various properties of macrophages which are inflammatory cells. For this reason, its effect is not remarkable, and almost no effect is exhibited to chronic inflammatory diseases in particular. On the other hand, the immunomodulator of the present invention controls the redox condition of macrophages, and can suppress the production of large number of harmful inflammatory mediators at the same time at once. In this context, the conventional concept to date of antiinflammatory drugs is said to be fundamentally changed.

As stated above, the useful pharmaceutical effect of the immunomodulator of the present invention in actual medical care is self-evident from its profitable immunological activity. It is useful for both the acute and chronic stages of diseases. Especially, it can widely be applied to diseases associated with the abnormal Th1/Th2 balance or the functional deficiency or the abnormality of macrophages, for example, cachexia of patients suffering from cancers, diabetes, chronic rheumatoid arthritis, autoimmune diseases such as SLE, chronic inflammatory diseases such as hepatitis, hepatic cirrhosis and inflammatory bowel diseases, and allergic diseases such as asthma, cutaneous atopy and sarcoidosis. It is also effective for chemoprevention of cancers. With respect to the cachectic condition of patients suffering from cancers, especially an effect to increase the survival rate is expected, and the immunomodulator is considered to be also useful in the improvement of quality of life (QOL) of the patient.

The administration form is not particularly limited, and it includes administration by injection and oral administration. However, the oral administration is advantageous. The dose of the substance having an activity of changing the content of reductive glutathione as an active ingredient is selected depending on the conditions of patients or the like to which the substance is administered or the use purpose. In the case of patients suffering from serious diseases, for example, a advanced gastric cancer, the dose is between 1 and 5,000 mg (oral drug), preferably between 10 and 500 mg/day. It is not particularly difficult to produce preparations, and preparations can be produced in the form of an oral agent, an injection, a percutaneous agent and the like as required in a usual manner.

It has been described above that the immunomodulator of the present invention is quite useful and quite new as a drug in a narrow sense. Since the immunomodulator of the present invention contains a substance capable of oral intake as a main ingredient, its use is not limited to drugs in actual medical care. That is, the immunomodulator of the present invention can also be provided in the form of a food (including all that are put into the mouth, such as a chewing gum, a tooth paste and the like), as a food for medical care, a health food or a special sanitary food containing a substance having an activity of changing the content of reductive glutathione in human macrophage (including monocyte) cells either singly or as a mixture, as well as in the form of a nutrient or an infusion. These are also included in the present invention. It can also be contained in a liquid component or take the form of a solid food.

The food, the nutrient and the infusion can be applied to the same diseases as those to which the drugs are applied.

The immunomodulator of the present invention can be provided in the form of the food, the nutrient and the infusion having an immunomodulatory function for improvement of the cachectic condition of patients suffering from cancers, diabetes, inflammatory bowel diseases, chronic rheumatoid arthritis, hepatitis and hepatic cirrhosis and for chemoprevention of cancer. The dose of the active ingredient may be determined according to what has been described in the above-mentioned drugs. It can be applied not only to patients suffering from attacked or chronic diseases but also to high-risk persons suffering from adult diseases or the like.

Having generally described this invention, a further understanding can be obtained by reference to certain specific examples which are provided herein for purposes of illustration only and are not intended to be limiting unless otherwise specified.

### EXAMPLES

### Example 1

### Test for Functions of Oxidative Macrophages and Reductive Macrophages

Oxidative macrophages were induced by administering 20 μg of LPS (lipopolysaccharide) to an abdominal cavity of a mouse, and reductive macrophages were induced by administering 100 μg of lentinan to an abdominal cavity of a mouse three times every two days. These were clarified by adhering peritoneal exudate cells to the plastic surface, then reacting the same with 10 μM of monochlorobimane at 37° C. f or 30 minutes and conducting analysis with Adherent Cell Analyzing System (ACAS). The increase in the amounts of oxidative macrophages can easily be measured visually from the fact that almost no reaction product is observed, that is, gray or blue image is obtained, and the increase in the amounts of reductive macrophages from the fact that the red or yellow image is obtained, respectively.

Accordingly, NO, IL-6 and PGE2 produced by inducing the peritoneal exudate cells into oxidative and reductive cells were measured.

(1) Materials

Cells: The peritoneal exudate cells obtained by the above-mentioned stimulation, namely, the macrophages were added to a 96-well microplate in an amount of 1×10⁵ cells/200 μl each.

Medium: Phenol red-free RPM 11640: 200 μl/well

LPS: Lipopolysaccharide (made by Sigma Co.) (origin: E. coli) 100 mg/ml

IFNγ: 100 units/ml

(2) Incubation

Incubated in a 5% CO₂ incubator at 37° C. for 48 hours.

(3) Measuring Method

After the completion of the above-mentioned incubation, the culture supernatant was recovered. The amount of IL-6 was measured by the proliferation assay using an IL-6 dependent cell line, MH60, the amount of PGE2 was measured using an ELISA-kit, and the amount of NO was measured using a Griess-Romijn reagent. These measurements were conducted by a method which those skilled in the art usually employ.

Results:

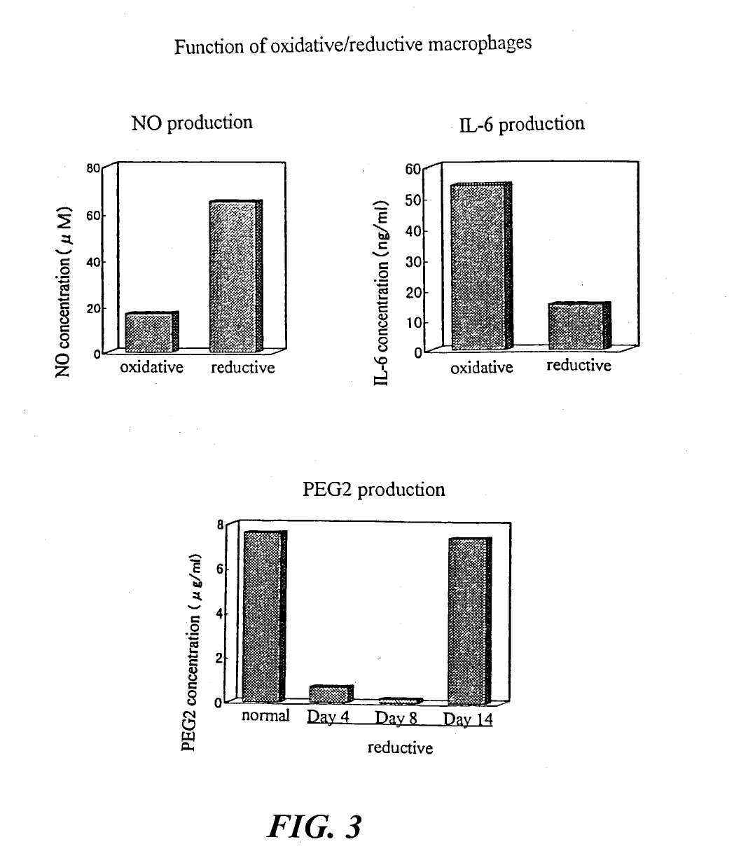

The results are shown in FIG. 3. As is clear from FIG. 3, there are differences in the concentration and the type among inflammatory cytokine IL-6, inflammatory mediator PGE2 and NO produced between oxidative macrophages and reductive macrophages. That is, with the oxidative macrophages, the production of IL-6, a Th2 cytokine and the production of PGE2 which is immunosuppressive to suppress the Th1 induction are increased, and the production of NO is decreased. On the contrary, with the reductive macrophages, the production of NO is increased, and the production of PGE2 and the production of IL-6 are suppressed. Thus, there is a functional difference between both macrophages.

### Example 2

### Test Using Animal Disease Models Which are Immunologically Deficient by Knocking Out a Gene

In order to clarify the mechanism underlying the conversion of an acute to a chronic phase and progression of inflammatory diseases, it is important to analyze molecularly why there is a difference in the production of an inflammatory mediator or a cytokine between oxidative MΦ and reductive MΦ. Generally, extracellular stimulation (ligand or the like) of the cell is signaled into cells through a receptor present on the cell surface. Various kinases are activated with signals from the receptor, and transcription factors are also activated in cytoplasm. The activated transcription factors are translocated into the nucleus, and bound to target genes to conduct gene expression. According to the recent studies, it is being clarified that the intracellular redox system regulates activation of transcription factors, translocation thereof into the nucleus and binding with genes (Annual Rev. Immunology, vol. 8, pp. 453-475, 1990; Embo J., 10, 2247-2251, 1991). It is currently unknown how the intracellular redox system participates in the gene expression system after the receptor triggering in MΦ. As a method of clarifying the same, MΦ was harvested from a knock out mouse deficient in a molecule participating in a signal transduction system from a receptor, and the function of the redox state was analyzed. Specifically, a common γ chain (γc) which is commonly used as a receptor constituting molecule of IL-2, IL-4, IL-7, IL-9 and IL-15 and Jak3 which is a molecule present downstream thereof and transducing a signal from γc were used as target molecules.

Cytokine and Stimulator:

As mouse IFNγ, a recombinant supplied by Genzyme was used. As human IL-2 and human IL-6, recombinants supplied by Ajinomoto Co. Inc. were used. As human IL-12, a recombinant supplied by Pharmingen was used.

As LPS, a substance derived from E. coli 055:B5 supplied by Difco was used. As lentinan, a preparation produced by Ajinomoto Co. Inc. was used.

Mice Used:

γc Knock out mice were obtained from Professor Sugamura, Tohoku University Medical School. Jak3 knock out mice were obtained from Professor Saito, Chiba University Medical School.

As wild mice used for mating and as a control, C57BL/6 obtained from Charles River Japan (CRJ) was used.

Harvest of Peritoneal MΦ:

Peritoneal cells were harvested by injecting 5 ml of a phenol red-free DMEM medium (supplied by Nikken Seibutsusha) ice-cooled into a peritoneal cavity of a mouse which had been put to sacrificial death with ether using an injection cylinder fitted with a 22-gauge needle, squeezing the same and pulling out the medium.

Determination of the Amount of IL-6:

A stimulator was added to 1×10⁵ MΦ, and the incubation was conducted at 37° C. for 2 days in a CO₂ incubator. After centrifugation, the culture supernatant was collected.

The amount of IL-6 was determined using IL-6 dependent mouse hybridoma MH60 cells (J. Eur. Immunol., vol. 18, p. 951, 1988). One hundred microliters of the culture supernatant were added to 100 μl of the MH60 cell suspension adjusted to 1×10 cells/ml in a 10% FCS-containing RPMI medium, and the mixed solution was incubated at 37° C. for 2 days in a CO₂ incubator. Subsequently, 10 μl of MTT (supplied by Sigma Co.) solution adjusted to a concentration of 5 mg/ml in the same medium were added thereto, and the reaction was conducted at 37° C. for 5 hours. After the completion of the reaction, the centrifugation was conducted. The supernatant (160 μl) was removed, and 100 μl of a mixture of hydrochloric acid and propanol were added to the residue. The suspension was conducted using a pipetman to dissolve the cells. Immediately after the dissolution, an absorbance of 570 nm was measured with an immunometer (supplied by Bio-Rad).

Measurement of a Concentration of NO₂:

A stimulator was added to 1×10 MΦ, and the incubation was conducted at 37° C. for 2 days in a CO₂, incubator. After the completion of the centrifugation, the culture supernatant was collected.

One hundred microliters of a Griess-Romijn reagent (supplied by Wako Pure Chemical Industries, Ltd.) adjusted to a concentration of 50 mg/ml in distilled water were added to 100 μl of the culture supernatant, and the reaction was conducted at room temperature for 15 minutes. After the completion of the reaction, an absorbance of 540 nm was measured. NaNO₂ was used as a standard.

Determination of GSH in Cells with ACAS:

Three-hundred microliters of a cell suspension adjusted to a concentration of 3×10⁵ cells/ml in an RPMI 1640 medium (phenol red-free) were charged into a chambered cover glass (#136439, supplied by Nunc), and incubated at 37° C. for 2 hours using a CO₂ incubator. The culture solution was washed with the same medium, and 300 μl of monochlorobimane (supplied by Molecular Probe) adjusted to 10 μM in the same medium were added thereto. The mixture was charged into a CO₂ incubator of 37° C., and the reaction was conducted for 30 minutes. The fluorescent intensity was measured with ACAS. In ACAS, a UV laser was used.

Determination of an Amount of IL-12:

The amount of IL-12 was determined through bioassay using cells of human T cell line 2D6 (J. Leukocyte Biology, vol. 61, p.346, 1997).

2D6 cells which had been incubated in an RPMI 1640 medium containing 500 μg/ml of recombinant human IL-12, 50 μM of 2-mercaptoethanol and 10% FCS (fetal calf serum) were moved to a tube, and centrifugally washed three times with the abovementioned medium without IL-12 and cell density was adjusted to 1×10/ml . The cell suspension was added in an amount of 100 μl each to a 96-well flat bottom plate containing a sample serially diluted in advance with an RPMI 1640 medium containing 50 μM of 2-mercaptoethanol and 10% FCS in an amount of 100 μl each. Subsequently, the mixture was charged into a 5% CO₂ incubator of 37° C., and incubated for 48 hours. For final 6 hours, ³H-TDR was pulsed (a substance adjusted to 370 kBq/ml in an RPMI 1640 medium containing 50 μM of 2-mercaptoethanol and 10% FCS was added in an amount of 50 μl each). The cells were harvested, and the radioactivity was measured using a P counter (Matrix 96, supplied by Packard).

Measurement of the GSH content in MΦ produced from knock out mice:

Peritoneal cells were produced from knock out mice, and the GSH content in cells was analyzed by ACAS using an MCB reagent. The content of reductive glutathione was clearly decreased in any mice compared with control mice (C57BL/6).

Function of MΦ Produced From Knock Out Mice:

Peritoneal cells were produced from wild mice (CS7BL/6) and knock out mice, and stimulated with LPS, IL-2, IFNγ and a combination thereof. The NO production, the IL-6 production and the IL-12 production were measured. Almost no NO production was observed in any mice derived MΦ in the absence of stimulation. In the stimulation with the combination of LPS and IFNγ, almost no additive effect was observed in the γc knock out mice, and the NO production was decreased to less than half that in control mice. The same results as in γc were provided in γc knock out mice. Further, the IL-6 production was analyzed. In the LPS stimulation, an increase in the IL-6 production was observed in γc knock out mice (962 pg/ml relative to 81 μg/ml of a control). In the IFNγ stimulation, an increase in the IL-6 production was observed in γc knock out mice. The results were the same with the suppression pattern of the NO production. Still further, the IL-12 production with the LPS stimulation and the IFNγ stimulation was examined. No production was observed at all in any mice derived MΦ. This proves that in the sick animals of the gene knock out mice used herein, the amount of the oxidative macrophages is increased to increase the humoral immunity or the allergic reaction mainly caused by Th2 and to decrease the cellular immunity supported by Th1. In the animal disease models, it is clearly shown that the diagnosis of immunological diseases required for the immunomodulator of the present invention is original and significant.

### Example 3

Determination of the amount of reductive glutathione in MΦ of advanced tumor-bearing mice

Method:

Oxidative and reductive macrophages collected from peritoneal cavities of advanced tumor-bearing cachectic mice (COLON 26) and normal mice were determined. The COLON 26 transplantable tumor, well known to induce a cancer cachexia was implanted subcutaneously in the back portion of CDF1 mice at a density of 5×10⁵ cells/mouse. On day 21 after the tumor implantation, the cachectic condition was provided. Five milliliters of a physiological saline solution were intraperitoneally injected into the mice which became resistant to a therapeutic-treatment. Peritoneal macrophages were collected, and suspended in a phenol red-free RPMI 1640 medium containing 10% fetal calf serum to a density of 3×10⁵ cells/ml. One hundred microliters of the suspension were charged in a Lab-Tek Chamber Slide (#136439, supplied by Nunc), and incubated in 5% CO₂ at 37° C. for 3 hours. After the nonadherent cells were removed, 200 μl of the above-mentioned medium free from serum were added thereto, and monochlorobimane (MCB) was added thereto in an amount of 10 μM. The reaction was conducted for 30 minutes, and the image analysis was conducted on the basis of the UV absorption using an ACAS device (supplied by Meridien).

Results:

The content of reductive glutathione was determined by ACAS. As a result, in the advanced tumor-bearing mice, the amount of the macrophage of which the reductive glutathi one content was decreased, namely, the oxidative macrophage was relatively increased in comparison with that in the normal mice. Since the amount of the oxidative macrophage was increased, the amount of IL-6 in the above-mentioned macrophage culture supernatant was markedly increased (600 pg/ml relative to 120 pg/ml in control mice). Further, the amount of PGE 2 was 32 ng/ml relative to 7.6 ng/ml in control mice, and it was increased to 5 times or more. It was found that the immunosuppressive state or the cachectic state at the advanced tumor-bearing stage is based on the excessive production of these mediators. In addition, the increase in an amount of active oxygen produced was also observed. It shows that the redox state of macrophages is measured upon determination of the glutathione content without measuring a large number of parameters whereby the examination for diagnosis of the pathological state and the immunological function of patients suffering from cancers can be conducted easily and exactly. Accordingly, the above-mentioned classification of macrophages enables the examination for diagnosis of diseases and the immunological function of patients suffering from cancers.

### Example 4

### Induction of Reductive Macrophages by Oral Administration of Glutathione Ethyl Ester to Advanced Tumor-Bearing Mice

The COLON 26 transplantable tumor was implanted subcutaneously in the back portion of CDF 1 mice at a density of 5×10⁵ cells/mouse. On day 21 after the tumor implantation, the mice were proved to be in the cachectic condition. Glutathione ethyl ester was orally administered to the mice every day in a dosage of 1 mg/0.5 ml/h. This oral administration was continued for 10 days. The peritoneal cells were collected from the mice in the same manner as in Example 3. Peritoneal macrophages were collected, and suspended in a phenol red-free RPMI 1640 medium containing 10% fetal calf serum to a density of 3×10⁵ cells/ml. The suspension was charged into a Lab-Tek Chamber Slide (#136439, supplied by Nunc) in an amount of 100 μl, and the incubation was conducted in 5% CO₂ at 37° C. for 3 hours. After the nonadherent cells were removed, 200 μl of the above-mentioned medium free from serum were added thereto, and 10 μM of monochlorobimane were then added thereto. The reaction was conducted for 30 minutes, and the image analysis was conducted based on the UV absorption using an ACAS device (supplied by Meridien).

Results:

The content of reductive glutathione was determined by the ACAS method. Consequently, in the advanced tumor-bearing model mice to which glutathione ethyl ester had been administered, the amount of the macrophage of which the reductive glutathione content was decreased, namely, the oxidative macrophage was relatively decreased in comparison with that in control mice to which the physiological saline solution had been administered. Since the amount of the reductive macrophage was increased, the amount of IL-6 in the above-mentioned macrophage culture supernatant was decreased (642 pg/ml relative to 5,200 pg/ml in control mice). Further, the amount of PGE2 was also much decreased to 6.5 ng/ml relative to 32 ng/ml in control mice. It was thus clarified that the immunosuppressive state or the cachectic state at the advanced tumor-bearing stage can be improved by the oral administration of glutathione ethyl ester. Accordingly, the average number of survival days of mice in the treated group increased from 42 (in control mice) to 148.

### Example 5

### Examination of Macrophages Collected From the Patient Suffering From Sarcoidosis and Conversion of Oxidative Macrophages to Reductive Macrophages

The amounts of oxidative and reductive monocytes macrophages contained in monocytes preparation separated and collected in a usual manner from the peripheral blood and the thoracic cavity of the patient suffering from sarcoidosis were examined by biochemically measuring the contents of reductive glutathione (GSH) and oxidative glutathione (GSSG) by the enzyme recycling method. The peripheral blood of the healthy person was used as a control.

Materials:

The peripheral blood of the healthy person and the peripheral blood of the patient suffering from sarcoidosis were collected with heparin. Or 150 ml of a physiological saline solution was injected into the bronctila of the patient using a bronchofiber, and 75 ml of bronchoalveolar lavaged fluid were recovered. Monocytes obtained by separating and purifying both of them using Ficoll-Hypaque (LYMPHOPREP) were suspended in an RPMI 1640 medium containing 10% fetal calf serum, and washed three times to obtain macrophage/monocyte preparation adherent to a glass petri dish for 30 minutes. Subsequently, a group incubated for 3 hours with the addition of 5 mM N-acetylcysteine (NAC) and a group of a medium component alone were prepared. A rubber policeman was used to recover adherent cells from the petri dish. With respect to 5×10⁵ macrophages, the following examination was conducted.

Method:

The contents of reductive glutathione and oxidative glutathione were measured by the above-mentioned enzyme recycling method.

Production of Samples:

One hundred microliters of Triton X-100 prepared with a 0.1 M phosphate buffer (pH 7.5) containing 5 mM EDTA ice-cooled were added to cell pellets which had been washed with PBS, and the mixture was allowed to stand at room temperature for 5 minutes to dissolve the cells. Fifteen microliters of 0.1 M HCl were added thereto, and 15 μl of a 50% sulfosalicylic acid (SSA) solution were further added thereto. The mixture was centrifuged at 12, 000 rpm for 5 minutes, and the supernatant was collected [*] to form a measuring sample having a total glutathione concentration (GSH+GSSG).

Measuring Method:

A 10 mM phosphate buffer (590 μl, pH 7.5) containing 0.5 mM EDTA, 100 μl of glutathione reductase (supplied by Boehlinger Mannheim) adjusted to a concentration of 6 u/ml in the same buffer, 50 μl of 4 mM NADPH (supplied by Sigma Co.) prepared with 5% NaHCO₃ and 10 μl of the sample were mixed. The mixture was incubated at 37° C. for 5 minutes. Fifty microliters of a 10 mM 5,5′-dithio-bis(2-nitrobenzoic acid) (DTNB, supplied by Sigma Co.) prepared with a 0.1 M phosphate buffer (pH 7.5) containing 5 mM EDTA were added thereto. The change in the absorbance of 412 nm at 37° C. over the course of time was measured using a spectrophotometer. As a standard sample, GSH (supplied by Sigma Co.) prepared in the same manner as the above-mentioned sample was used. Separately, the content of oxidative glutathione (GSSG) alone was measured (—), and 2 μl of 2-vinylpyridine (supplied by Tokyo Kaseisha) were added thereto after the above-mentioned procedure (*). These were mixed at room temperature for 1 minute, and the pH was adjusted to 7.5. Subsequently, the reaction mixture was allowed to stand at room temperature for 60 minutes to form a measuring sample. The measurement was conducted in the above-mentioned manner (—). The content of reductive glutathione (GSH) was obtained by the subtraction from the total glutathione content.

Results:

With respect to the contents of reductive glutathione and oxidative glutathione in the peripheral blood of the patient, the GSSG content was 5.29 μM, and the GSH content was 20.45 μM. Thus, the ratio of reductive GSH was approximately 80%, and still higher (the ratio of reductive GSH was 90% or more in the healthy person). In the macrophages within the thoracic cavity, the content of reductive GSH was 1.45 μM, and the content of oxidative GSSG was 15.85 μM. Thus, the ratio of oxidative GSSG was approximately 86%, and the presence ratio thereof was completely inverted. In the NAC addition group, the content of reductive GSH was 20.45 μM, and the content of oxidative GSSG was 4.32 μM. Thus, the content of oxidative GSSG was largely decreased, and the ratio of reductive GSH exceeded 80%. In this manner, the peripheral blood level was recovered. It shows that in this disease, the oxidative macrophages play a great role in the progression of the disease and this progression can be improved through NAC administration. Thus, the usefulness of the present invention is demonstrated.

### Example 6

### Induction of Reductive Macrophages by Oral Administration of NAC and GSH-OEt

Macrophages (MΦ) were prepared from knock out mice deficient in a molecule participating in a signal transduction system from a receptor, and the function of the redox system was analyzed. Specifically, a common γ chain (γc) which is commonly used as a receptor constituting molecule of IL-2, IL-4, IL-7, IL-9 and IL-15 and JaK3, a molecule present downstream thereof and transducing a signal from γc were gene targeted molecules. Methods applied in Example 2 was repeated. JaK3 knock out mice were divided into three groups. A control group was a group of usual city water free-intake. An NAC group was a group of free intake of city water containing 1 mg/ml of NAC. A GSH-OEt group was a group of free intake of city water containing 1 mg/ml of GSH-OEt. Breeding was continued under the SPF condition for 24 days, and peritoneal exudate cells, namely, macrophages were likewise obtained.

Cytokines and Stimulator:

A recombinant supplied by Genzyme was used as mouse IFN. Recombinants supplied by Ajinomoto Co., Inc. were used as human IL-2 and human IL-6. A recombinant supplied by Pharmigen was used as human IL-12.

A product derived from E. coli. 055; B5 as supplied by Difco was used as LPS. A preparation supplied by Ajinomoto Co. Inc. was used as lentinan.

Determination of an amount of IL-6:

Measurement of NO₂ concentration:

Determination of a GSH content in cells by ACAS:

Determination of amount of IL-12:

These were all conducted in the same manner as in Example 2.

Measurement of a GSH content in MΦ prepared from knock out mice:

Peritoneal cells of knock out mice which had undergone the respective treatments were harvested, and the GSH content in the cells was analyzed using ACAS with MCB. In any of the mice in the groups of free intake of city waters containing NAC and GSH-OEt, the content of reductive glutathione in MΦ was markedly increased in comparison with that in control mice (city water free intake group).

Function of MΦ Harvested from Knock Out Mice

Peritoneal cells were harvested from three groups of knock out mice, and stimulated with LPS, IL-2, IFNγ and a combination thereof. The NO production, the IL-6 production and the IL12 production were measured. With respect to the NO production, almost no NO production was observed in any mice derived MΦ in the absence of stimulation. Then, the IL-6 production was analyzed. In LPS stimulation, the amount was detected as 962 pg/ml in the knock out mice derived MΦ culture, 122 pg/ml in the NAC group, and 82 pg/ml in the GSH-OEt group. In view of the function, it was identified that the conversion to reductive macrophages was possible. In consideration of the fact that IL-6 is a main cytokine of inducing Th2, it is clearly shown that the biological Th1/Th2 balance can be controlled by the oral intake of these substances. This result was inversely related with the suppression of the NO production and the recovery pattern with medications. Next, the IL-12 production by stimulation of LPS or IFNγ was examined. No production was observed at all in the control group. This shows that in the animals disease models, the JaK3 gene knock out mice used here, the amount of the oxidative macrophage is increased, humoral immunity or an allergic reaction mainly caused by Th2 is increased, and cellular immunity caused by Th1 is decreased. On the other hand, it was identified that in the NAC and GSH-OEt administration groups, the amounts of IL-12 are 420 pg/ml and 610 pg/ml respectively. This proves that in the animal disease models, the immunomodulator of the present invention is also useful in the improvement of the immune diseases, and is original and significant.

### Example 7

### Difference in the IL-12 Production Between Reductive and Oxidative Macrophages

When there are defects in differentiation, selection and functional expression steps of T cells, the host immune system becomes deficient. From this fact, it is considered that T cells play an central role in the host immune system. Helper T cells (Th) which are one subset of T cells produce lymphokines to regulate immunocytes or inflammatory cells. Recently, the following concept has been proposed. That is, Th is further classified into two types, Th1 and Th2 depending on the types of the lymphokines produced, and these cells have the different immunological functions (J. Immunol., vol. 136, pp. 2348, 1986). That is, Th1 produces IL-2 or IFNγ, and is a main cell to modulate cellular immunity. Th2 produces IL-4, IL-5, IL-6 and IL-10, and is a main cell to modulate humoral immunity. The homeostasis of the in vivo immunity is maintained by the Th1/Th2 balance. Usually, when the Th1/Th2 balance is inclined to either of Th1 and Th2, the host responds by attempting to correct the skewing and tend to maintain the homeostasis. However, it is considered that when the imbalance is not corrected for some reasons, immunological diseases will occur. Th1 and Th2 are differentiated from the precursor of them, namely Th0. In the differentiation of Th0 to Th1, IL-12 produced by MΦ is important (Immunology Today, vol. 335, p 14, 1993). In the differentiation of Th0 to Th2, IL-4 produced by NKT cells is important (J. Exp. Medicine, vol. 179, pp. 1285, 1994).

In the above-mentioned Example, it is clarified that the MΦ function differs depending on the difference in the redox state of MΦ. With respect to MΦ, there are two types of MΦ, oxidative MΦ and reductive MΦ based on the difference in the GSH content, and these two distinctive MΦ behave differently in the NO or IL-6 production. The main producer of IL-12, which induces differentiation of Th0 to Th1 and which is a key molecule of controlling the Th1/Th2 balance, is considered to be MO. However, the detailed analysis has not yet been reported. In view of the clarification of attack mechanism of immunological diseases, it is quite interesting to know whether or not the IL-12 production is different between oxidative MΦ and reductive MΦ. The present inventors have found that IL-12 is produced from only reductive MΦ, and that IL-4 considered to control the Th1/Th2 balance like IL-12 acts on oxidative MΦ and reductive MΦ whereby the Th1/Th2 balance is skewed to the Th2 side. On the basis of the findings which were obtained prior to the completion of the present invention, it is shown that the redox state of MΦ regulates the Th1/Th2 balance, and the usefulness of the present invention in the diagnosis of immune diseases is described.

IL-12 is produced from reductive MΦ:

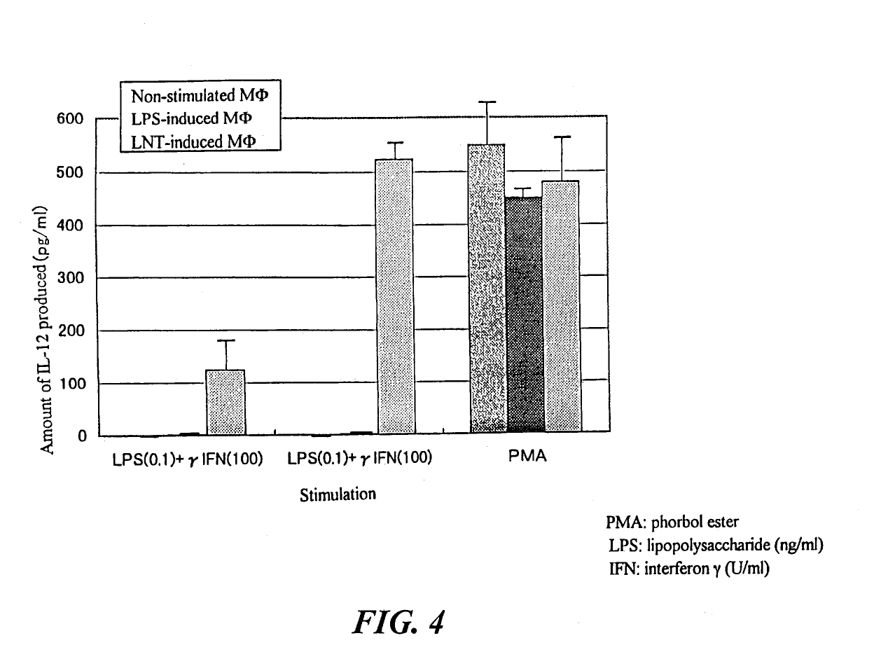

In Example 1, it was indicated that MΦ produced by injecting lentinan (LNT) intraperitoneally was reductive MΦ with the high GSH content and that MΦ induced by injecting LPS intraperitoneally was oxidative MΦ with the low GSH content. It was examined whether there is a difference in the IL-12 production between LNT-induced MΦ and LPS-induced MΦ. By stimulation with LPS and INFγ, the remarkable TL-12 production (1,312 pg/ml) was observed in the LNT-induced MΦ. However, no IL-12 production was observed in LPS-induced MΦ and control resident MΦ(FIG. 4). Next, the same analysis was conducted using MΦinduced by intraperitoneally injecting substances for changing the GSH content in cells. With respect to MΦ induced by administering GSH-OEt, a substance increasing the GSH content in cells and DEM, a substance decreasing the same, IL-12 (3,570 pg/ml) was produced only in MΦ derived from GSH-OEt-administered mice through stimulation with LPS and IFNγ. These results show that IL-12 is produced only by reductive MΦ having the high GSH content in cells.

The IL-12 production from reductive MΦ is suppressed by decreasing the GSH content in cells:

As mentioned above, IL-12 was produced only in reductive MΦ having the high GSH content in cells. It was examined whether this production is suppressed by converting MΦ to oxidative MΦ. That is, it was analyzed whether the IL-12 production is suppressed by exposing lentinan-induced MΦ with DEM. As a result, it was clarified that the IL-12 production (828 pg/ml) from lentinan-induced MΦ is completely suppressed (0 pg/ml) with the addition of DEM. That is, it was suggested that reductive glutathione in cells is deprived through DEM treatment and reductive MΦ is converted to oxidative MΦ to suppress the IL12 production.

IL-4 suppresses the IL-12 production by reductive MΦ:

IL-4 is a cytokine which acts on MΦ suppressively. IL-4 is considered to have an opposite function to IL-12 in the Th1/Th2 balance as well. Accordingly, it was examined whether IL-4 acts suppressively on the IL-12 production by reductive MΦ. It was clarified that the IL-12 production by LNT-induced M<and the IL-12 production by GSH-OEt administered mouse MO are remarkably suppressed by the pretreatment with IL-4 (from 1,580 pg/ml to 370 pg/ml and from 490 pg/ml to 258 pg/ml). That is, it was suggested that there is a possibility that IL-4 acts on MΦ to suppress the IL-12 production whereby the Th1/Th2 balance is skewed to the Th2 side. At this time, it was clarified from the image analysis by ACAS that IL4 markedly decreases the content of reductive glutathione in MΦ.

IL-4 suppresses the No production and increases the IL-6 production:

Reductive MΦ increases the NO production by the IFNγ stimulation in comparison with oxidative MΦ, and rather suppresses the IL-6 production. IFNγ is known to be a cytokine produced from Th1 cells. What function IL-4 shows in the NO production and the IL-6 production with IFNγ was analyzed using respective MΦ's. IFNγ NO production from MΦ pretreated with IL-4 was significantly suppressed in comparison with MΦ untreated with IL-4. Further, MΦ of which the GSH content in cells was increased by the stimulation with GSH-OEt and MΦ of which the GSH content in cells was decreased by the stimulation with DEM were pretreated with IL-4, and IFNγ thereafter and LPS stimulation was carried out to induce NO production. As a result, the NO production was remarkably suppressed in IL-4 treated MΦ in comparison with IL-4-untreated MΦ in both subsets of MΦ.

Meanwhile, with respect to the IL-6 production, the production with IFNγ was markedly increased by pretreatment with IL-4 in resident MΦ, LPS-induced MΦ and LNT-induced MΦ. Further, MΦ of which the GSH content in cells was increased by the stimulation with GSH-OEt and MΦ of which the GSH content in cells was decreased by the stimulation of DEM were pretreated with IL-4, and IFNγ was exposed thereon to induce the IL-6 production. Consequently, the IL-6 production was increased in IL-4-treated MΦ's in comparison with IL-4-untreated MΦ. These results revealed that IL-4 induces oxidative macrophages by decreasing the content of reductive glutathione in cells, suppressing the NO production by the stimulation with IFNγ and increasing the IL-6 production. This indicates that IL-4 suppresses the NO production by IFNγ, namely, Th1 type response, increases the IL6 production by IFNγ, and has an activity of enhancing Th2 type response. These findings scientifically prove the usefulness of the immunomodulator according to the present invention.

### Example 8

### Enhancement of IL-12 Production with a Combination of NAC Orally Taken in and IL-2 Infusion

Two groups, namely, a group of 8-week-old DBA/2 female mice which were caused to freely drink city water as in Example 6, and a group of the same female mice which were caused to freely drink city water containing 1 mg/ml of NAC, were prepared. Further, the two groups of the above-mentioned mice to which human recombinant IL-2 in an amount of 2 μg/0.5 ml/h was intraperitonerally administered twice a day, every two days for two weeks were provided. On day 14, the IL-12 production from MΦ was measured in the same manner as in Example 6.

Measurement of the GSH content in MΦ prepared:

The peritoneal cells were harvested from the mice which had undergone the respective treatments, and the GSH content in cells was analyzed by ACAS using an MCB reagent. In comparison with control mice (group caused to freely drink city water), the content of reductive glutathione was markedly increased in the group caused to freely drink NAC-containing city water and the IL-2 administration group, showing the image of reductive MΦ. The content of reductive glutathione was more increased in the group which had undergone the combination of the free-drinking of NAC-containing city water and the IL-2 administration than in any of the group of the free drinking of NAC-containing city water and the IL-2 administration group. Thus, the effect brought forth by the combination of the treatments in the induction of reductive MΦ was clearly observed in the ACAS image analysis. In the group which had undergone the combination of the treatments, the increase in the content of reductive glutathione was observed in all MΦ's (in contrast with the fact that the increase in the content thereof in the group of the sole treatment was observed in from 40 to 50% of MΦ).

Function of MΦ produced from each group:

Peritoneal cells were harvested from four groups of the mice, and stimulated with LPS and IFNγ. Then, the NO production, the IL-6 production and the IL-12 production were measured. Since the content of reductive macrophage was increased in three groups of the sole administration and the combined administration in comparison with the control group, the amount of IL-6 in the macrophage culture supernatant was decreased (relative to 1,240 pg/ml in control mice, 320 pg/ml in the group caused to freely drink NAC-containing city water, 520 pg/ml in the IL-2 administration group, and 67 pg/ml in the group which had undergone the combination of the-free-drinking of NAC-containing city water and the IL-2 administration). In consideration of the fact that IL-6 is a main cytokine inducing Th2, the combination of the NAC oral intake and the injection of IL-2, the cytokine can control the Th1/Th2 balance more strongly. The increase pattern of the No production was inversely related with the IL-6 production. With respect to the IL-12 production, the amount of IL-12 was 620 pg/ml in the group caused to freely drink NAC-containing city water, 946 pg/ml in the IL-2 administration group, and 2,386 pg/ml in the group which had undergone the free drinking of NAC-containing city water and the IL-2 administration in comparison with 0 pg/ml in control mice. Thus, the remarkable effect was observed by the combination of the treatments. It shows that the present invention provides the immunomodulator which is useful for the remarkable improvement of the immunological diseases in combination with the cytokines, and is therefore original and significant.

The immunomodulator of the present invention enables the novel control of the function of macrophages (including monocytes), and can treat, improve or prevent autoimmune diseases such as hepatic cirrhosis, hepatitis, diabetes, inflammatory bowel diseases, chronic rheumatoid arthritis, asthma and cutaneous atopy, allergic diseases, cancers and the like. Especially, this immunomodulator can be taken in orally, and it can be used as a drug, a food, a nutrient and an infusion.

Obviously, numerous modifications and variations of the present invention are possible in light of the above teachings. It is therefore to be understood that within the scope of the appended claims, the invention may be practiced otherwise than as specifically described herein.

This Application is based on Japanese Patent Application 9-312727, filed on Oct. 29, 1997, and incorporated herein by reference in its entirety.

1. An immunomodulator, comprising a substance having an activity of changing a intracellular content of reductive glutathione in macrophages.

2. The immunomodulator of claim 1, wherein said substance induces production of interleukin 12 by increasing the intracellular content of reductive glutathione in the macrophages.

3. The immunomodulator of claim 1, wherein said substance contains at least one antioxidant selected from the group consisting of glutathione precursors, glutathione derivatives, lipoic acid and derivatives thereof, ortene, flavonoid and derivatives thereof.

4. An immunomodulator composition, comprising at least one type of immunomodulator selected from the group consisting of β (1-3) glucans and cytokines in combination with the immunomodulator of claim 1.

5. An immunomodulator, containing a substance capable of selectively removing at least one of two types of macrophages, an oxidative macrophage and a reductive macrophage which are different in the intracellular content of reductive glutathione.

6. The immunomodulator of claim 5, wherein said substance is one in which a cytotoxic DNA alkylating agent is conjugated with glutathione or one which has cytotoxicity after being incorporated into macrophages as a precursor.

7. A food, a nutrient or an infusion containing the immunomodulator of claim 1.

8. A method of improving treating patients suffering from cancer cachexia, diabetes, inflammatory bowel diseases, chronic rheumatoid arthritis, pneumonia, hepatitis, hepatic cirrhosis and chemoprevention of cancers, comprising administering an effective amount of the immunomodulator of claim 1 to a patient in need thereof.

9. A method of categorizing macrophages, comprising:
measuring the concentration of reduced glutathione, GSH, in a macrophage, and
categorizing the macrophage as a reductive macrophage when the concentration of GSH in the macrophage is increased in comparison to a resident macrophage, or
categorizing the macrophage as an oxidative macrophage when the concentration of GSH in the macrophage is decreased in comparison to a resident macrophage.

10. The method of claim 9, wherein the concentration of GSH in the macrophage is measured with monochlorobimane.

11. The method of claim 9, wherein
the concentration of GSH in the reductive macrophage is at least twice as high as the resident macrophage, and
the concentration of GSH in the oxidative macrophage is at most ⅕ as high as the resident macrophage.

12. A method of altering the macrophage content of a subject, comprising administering the immunomodulator of claim 1.
